(19) United States
(12) Patent Application Publication
(10) Pub. No.: US 2019/0118222 A1
(43) Pub. Date: Apr. 25, 2019

(54) ULTRASONIC TRANSDUCER, MANUFACTURING METHOD THEREOF, AND ULTRASONIC IMAGING DEVICE

(71) Applicant: HITACHI, LTD., Chiyoda-ku, Tokyo (JP)

(72) Inventors: Hiroaki HASEGAWA, Tokyo (JP); Shuntaro MACHIDA, Tokyo (JP); Taiichi TAKEZAKI, Tokyo (JP); Daisuke RYUZAKI, Tokyo (JP)

(21) Appl. No.: 16/090,948

(22) PCT Filed: Jul. 7, 2017
(86) PCT No.: PCT/JP2017/024949

§ 371 Date: Oct. 3, 2018

(30) Foreign Application Priority Data
Sep. 28, 2016 (JP) 2016-189023

(51) Int. Cl. B06B 1/02; A61B 8/00

(52) U.S. Cl. CPC B06B 1/0292; B06B 2201/76; A61B 8/4483

(57) ABSTRACT

An ultrasonic transducer includes: a hollow portion 110 formed between insulating films 104 and 106 interposed between a lower electrode 103 and an upper electrode 107 above a substrate 101; and a membrane 120 that is configured of insulating films 106, 108, 111, and 112 and the upper electrode 107 above the hollow portion 110 and vibrates at a time of transmission/reception of ultrasonic wave. Also, the hollow portion 110 has a cross-sectional shape according to which a relationship of h1>h2>0 is established when a thickness of a center portion is given as h1 and a thickness of an outer peripheral portion is given as h2.

### TECHNICAL FIELD

The present invention relates to an ultrasonic transducer, a manufacturing method thereof, and an ultrasonic imaging device using the same.

### BACKGROUND ART

An ultrasonic transducer element is incorporated in an ultrasonic probe (probe) of an ultrasonic imaging device, and is used for various purposes such as diagnosis of a tumor in a human body or inspection of a crack in a building, by transmitting and receiving ultrasonic wave.

Conventionally, a piezoelectric ceramic typified by lead zirconate titanate (PZT) is used as an electroacoustic conversion element of the probe of this type of ultrasonic imaging device, but in recent years, a capacitive detection ultrasonic transducer (Capacitive Micromachined Ultrasonic Transducer; hereinafter abbreviated as “CMUT”) having wider band characteristics than the piezoelectric ceramic is gaining attention and research and development thereof have been progressing.

According to a basic structure of the CMUT, a hollow portion (cavity) is provided in an insulating layer between a lower electrode and an upper electrode disposed above the lower electrode, and insulating layers and the upper electrode above the hollow portion are caused to function as a membrane (also referred to as “diaphragm”). At the time of transmission of ultrasonic wave, a DC voltage and an AC voltage are superimposed and applied between the upper electrode and the lower electrode, and the membrane is vibrated at a frequency of the AC voltage by an electrostatic force generated between the electrodes at that time. On the other hand, at the time of reception, the membrane is vibrated by pressure from ultrasonic wave that reaches a surface of the membrane, and a change in a distance between the electrodes caused at that time is electrically detected as a change in capacitance.

Patent Document 1 is for solving a problem of reduction in a transmission/reception efficiency due to the characteristics of the CMUT that a membrane near an outer peripheral portion of a hollow portion restrained by insulating layers is less easily displaced than the membrane near a center portion of the hollow portion, and discloses a technique of monotonically reducing a height (distance in a vertical direction) of the hollow portion in a curved manner from the center portion toward the outer peripheral portion and making the height of the hollow portion zero at the outer peripheral portion.

In the CMUT of Patent Document 1, the electrostatic force generated at electrodes can be increased by reducing the distance between the electrodes at the outer peripheral portion of the hollow portion (in the case where a dielectric is inserted, the distance equivalent to that obtained by conversion to vacuum based on a relative permittivity of the dielectric), and thus, a desirable effect that a drive voltage necessary to drive the membrane can be reduced is obtained.

### RELATED ART DOCUMENTS

### Patent Documents

Patent Document 1: International Patent Publication WO 13/065365 pamphlet

### SUMMARY OF THE INVENTION

### Problems to be Solved by the Invention

In the CMUT disclosed in Patent Document 1, when the membrane is vibrated at a maximum amplitude by a predetermined voltage, insulating layers above and below the hollow portion come into contact with each other not only at the center portion of the hollow portion, but also at the outer peripheral portion of the hollow portion, so that a contact area between the insulating layers is large and thus the amount of charges injected from the electrodes to the insulating layers is increased. Accordingly, in the case of use over a long period of time, charges between the upper and lower electrodes may be shielded due to accumulation of charges trapped in the insulating layers, so that appropriate driving is disabled or dielectric breakdown of the insulating layers is likely to occur, and there are issues to be solved in terms of reliability.

Therefore, in the CMUT having the structure described above, improvement has to be made to suppress the reduction in reliability due to excessive injection of charges from the electrodes to the insulating layers, in addition to reducing the drive voltage necessary to vibrate the membrane.

The above and other objects and novel features of the present invention will be apparent from the description of the present specification and the appended drawings.

### Means for Solving the Problems

The following is a brief overview of a representative embodiment in the embodiments disclosed in the present application.

A CMUT according to the representative embodiment includes: a hollow portion formed between two layers of insulating films interposed between a lower electrode and an upper electrode above a substrate; and a membrane that is configured of a plurality of insulating films and the upper electrode above the hollow portion and vibrates at a time of transmission/reception of ultrasonic wave, and the hollow portion has a cross-sectional shape according to which a relationship of h1>h2>0 is established when a thickness of a center portion is given as h1 and a thickness of an outer peripheral portion is given as h2.

### Effects of the Invention

According to the representative embodiment, a CMUT capable of achieving both a reduced drive voltage and improved reliability can be realized.

### BRIEF DESCRIPTIONS OF THE DRAWINGS

FIG. 1 is a plan view of main parts of a CMUT according to a first embodiment;

FIG. 2(a) is a cross-sectional view taken along line IIa-IIa in FIG. 1, and FIG. 2(b) is a cross-sectional view taken along line IIb-IIb in FIG. 1;

FIGS. 3(a) and 3(b) are cross-sectional views of main parts, showing an example of a manufacturing method of the CMUT according to the first embodiment;

FIGS. 4(a) and 4(b) are cross-sectional views of main parts, showing the manufacturing method of the CMUT following FIGS. 3(a) and 3(b);

FIGS. 5(a) and 5(b) are cross-sectional views of main parts, showing the manufacturing method of the CMUT following FIGS. 4(a) and 4(b);

FIGS. 6(a) and 6(b) are cross-sectional views of main parts, showing the manufacturing method of the CMUT following FIGS. 5(a) and 5(b);

FIGS. 7(a) and 7(b) are cross-sectional views of main parts, showing the manufacturing method of the CMUT following FIGS. 6(a) and 6(b);

FIGS. 8(a) and 8(b) are cross-sectional views of main parts, showing the manufacturing method of the CMUT following FIGS. 7(a) and 7(b);

FIGS. 9(a) and 9(b) are cross-sectional views of main parts, showing the manufacturing method of the CMUT following FIGS. 8(a) and 8(b);

FIGS. 10(a) and 10(b) are cross-sectional views of main parts, showing the manufacturing method of the CMUT following FIGS. 9(a) and 9(b);

FIGS. 11(a) and 11(b) are cross-sectional views of main parts, showing another example of the manufacturing method of the CMUT according to the first embodiment;

FIGS. 12(a) and 12(b) are cross-sectional views of main parts, showing the manufacturing method of the CMUT following FIGS. 11(a) and 11(b);

FIGS. 13(a) and 13(b) are cross-sectional views of main parts, showing the manufacturing method of the CMUT following FIGS. 12(a) and 12(b);

FIGS. 14(a) and 14(b) are cross-sectional views of main parts, showing the manufacturing method of the CMUT following FIGS. 13(a) and 13(b);

FIGS. 15(a) and 15(b) are cross-sectional views of main parts, showing the manufacturing method of the CMUT following FIGS. 14(a) and 14(b);

FIGS. 16(a) and 16(b) are cross-sectional views of main parts, showing the manufacturing method of the CMUT following FIGS. 15(a) and 15(b);

FIG. 17 is a graph describing a specific example of an influence on an electrostatic force due to an increase in an electrode area;

FIG. 18 is a graph describing an effect of the CMUT according to the first embodiment;

FIG. 19 is a cross-sectional view of main parts, showing an example of a specific measure taken against electric field concentration;

FIG. 20 is a cross-sectional view of main parts, showing another example of the specific measure taken against electric field concentration;

FIG. 21 is a perspective view showing an external appearance of an ultrasonic imaging device provided with the CMUT of the first embodiment; and

FIG. 22 is a block diagram showing functions of the ultrasonic imaging device shown in FIG. 21.

### DETAILED DESCRIPTION OF PREFERRED EMBODIMENTS

Hereinafter, embodiments of the present invention will be described in detail with reference to the drawings. Throughout the drawings for describing the embodiments, members having the same function are denoted by the same reference character, and repeated description thereof is omitted. In the embodiments, the same or similar portion will not be repeatedly described in principle unless it is necessary to do so. In the drawings used for describing the embodiments, hatching may be used even in a plan view so as to make a structure easily understood.

### First Embodiment

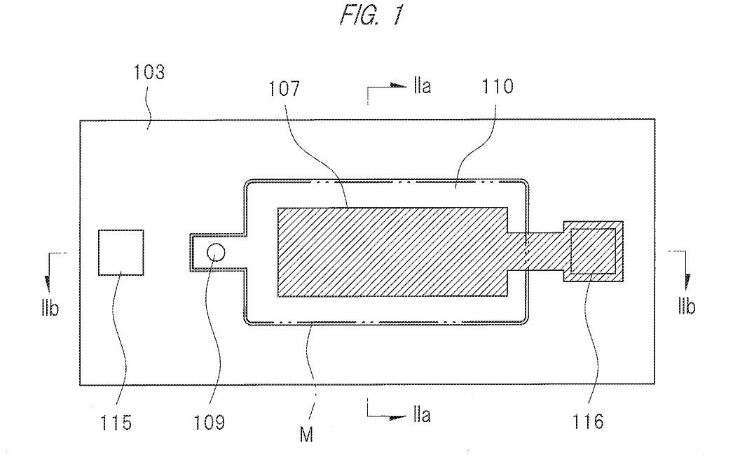

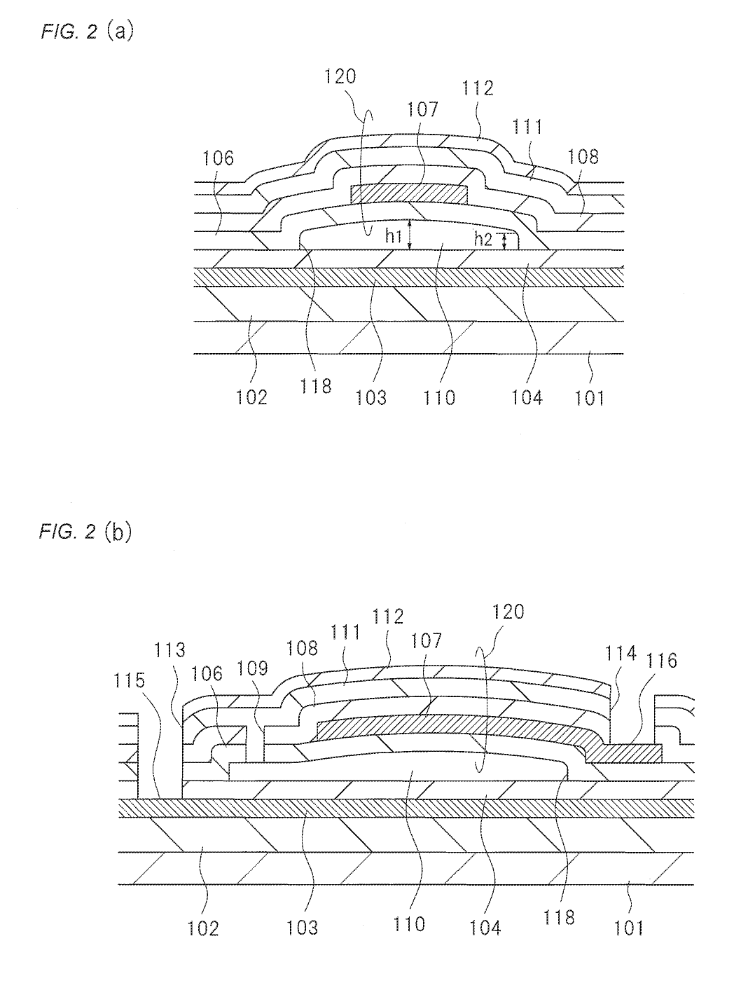

FIG. 1 is a plan view showing a region corresponding to one cell of a CMUT according to a present embodiment, FIG. 2(a) is a cross-sectional view taken along line IIa-IIa in FIG. 1, and FIG. 2(b) is a cross-sectional view taken along line IIb-IIb in FIG. 1. FIG. 1 mainly shows a planar layout of upper and lower electrodes and a hollow portion formed between the electrodes, and illustration of insulating films is omitted.

A cell of the CMUT includes an insulating film 102 formed over a substrate 101 made of monocrystalline silicon, a lower electrode 103 formed on the insulating film 102, two layers of insulating films 104 and 106 formed over the lower electrode 103, a hollow portion 110 configured of a void space formed between the insulating film 104 and the insulating film 106, an upper electrode 107 formed above the hollow portion 110 with the insulating film 106 interposed therebetween, and three layers of insulating films 108, 111, and 112 formed on an upper part of the upper electrode 107. Note that a protective film (not shown) for preventing adhesion of foreign matters, which is made of polyimide resin or the like, may sometimes be provided as necessary on an upper part of the insulating film 112 in an uppermost layer.

Parts of the insulating films 106, 108, 111, and 112 and the upper electrode 107 positioned above the hollow portion 110 (parts inside a boundary indicated by a two-dot chain line M in FIG. 1) function as a membrane 120 which is vibrated at the time of transmission/reception of ultrasonic wave. Also, parts of the insulating films 106, 108, 111, and 112, which surround the region that functions as the membrane 120 (parts surrounding the boundary M) function as a fixing portion for supporting the membrane 120.

A pad 115 for external connection formed of a part of the lower electrode 103 is exposed at a bottom portion of a connection hole 113 formed by forming an opening in the insulating films 104, 106, 108, 111, and 112, and a pad 116 for external connection formed of a part of the upper electrode 107 is exposed at a bottom portion of a connection hole 114 formed by forming an opening in the insulating films 108, 111, and 112. A DC voltage and an AC voltage are applied to the CMUT from an external power source through the pads 115 and 116. A reference character 109 in the drawing denotes an opening which is formed in the insulating films 106 and 108 in a step (described later) of forming the hollow portion 110.

The CMUT has a structure in which a large number of unit cells structured in the above manner are arranged on a main surface of the substrate 101, along one direction or two directions perpendicular to each other.

The hollow portion 110, which is provided in each unit cell, has a cross-sectional shape that is thicker at a center portion than an outer peripheral portion. Also, a side wall portion 118 formed along the outer peripheral portion is provided at the outer peripheral portion of the hollow portion 110. In other words, when a thickness (height) of the center portion is given as h1 and a thickness (height) of the side wall portion 118 provided at the outer peripheral portion is given as h2, the hollow portion 110 has a cross-sectional shape according to which a relationship of h1>h2>0 is established. The thickness (h1) of the center portion is preferably at least 1.5 times the thickness (h2) of the outer peripheral portion.

In the example shown in the drawing, the thickness of the hollow portion 110 is monotonically reduced in a curved manner from the center portion toward the outer peripheral portion, but the cross-sectional shape of the hollow portion 110 is not limited to such a shape, and a cross-sectional shape in which the thickness is approximately linearly reduced from the center portion toward the outer peripheral portion or a cross-sectional shape in which unevenness is locally present and the thickness is reduced in a curved manner from the center portion toward the outer peripheral portion may also be adopted.

In addition, the cross-sectional shape of the hollow portion 110 in the drawing has a flat bottom surface and a protruding upper surface, but the cross-sectional shape may have a recessed bottom surface and a flat upper surface. However, the cross-sectional shape shown in the drawing is desirable when taking into account ease of manufacturing.

Further, a planar shape of the hollow portion 110 in the drawing is a rectangle, but the planar shape of the hollow portion 110 is not limited to a rectangle, and may alternatively be a circle, an oval, or a polygon with five or more sides (hexagon, octagon), for example.

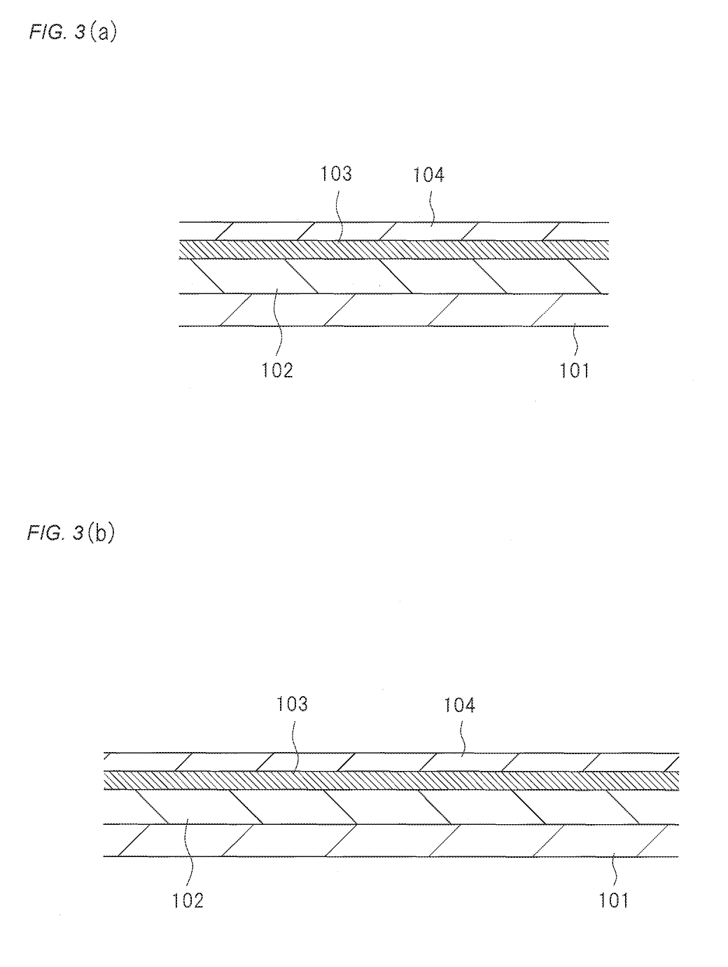

Next, an example of a manufacturing method of the CMUT of the present embodiment will be described with reference to FIGS. 3(a) to 10(b). In each of FIGS. 3(a) to 10(b), (a) is a cross-sectional view taken along line IIa-IIa in FIG. 1, and (b) is a cross-sectional view taken along line IIb-IIb in FIG. 1.

First, as shown in FIGS. 3(a) and 3(b), the insulating film 102 made of a silicon oxide film having a film thickness of about 500 nm is formed over the substrate 101 by the chemical vapor deposition (CVD) method or the thermal oxidation method, and then an aluminum alloy film having a film thickness of about 100 nm is deposited on an upper part of the insulating film 102 by the sputtering method to form the lower electrode 103. Subsequently, the insulating film 104 made of a silicon oxide film having a film thickness of about 200 nm is deposited on an upper part of the lower electrode 103 by the plasma CVD method.

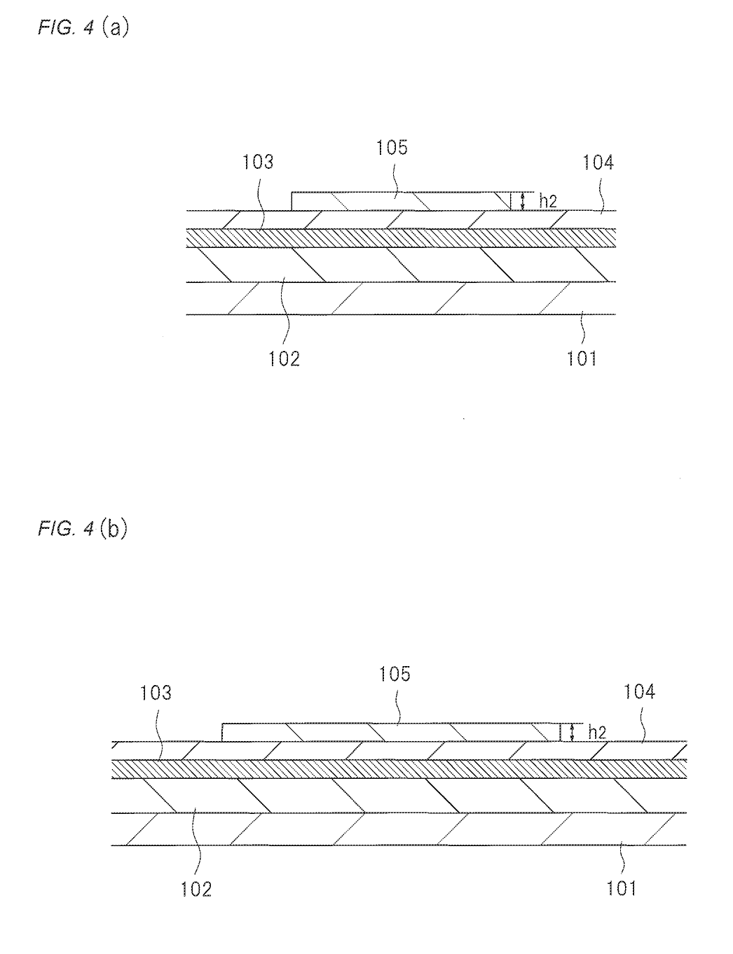

Next, as shown in FIGS. 4(a) and 4(b), a polycrystalline silicon film having a film thickness of about 100 nm is deposited on an upper part of the insulating film 104 by the plasma CVD method, and then the polycrystalline silicon film is patterned by using the photolithography technique and the dry etching technique, thereby forming a sacrificial layer (dummy layer) 105 made of the polycrystalline silicon film on the upper part of the insulating film 104. A region where the sacrificial layer 105 is formed is a region to be the hollow portion 110 in a later step, and a film thickness of the sacrificial layer 105 is equivalent to the thickness (h2) of the side wall portion 118 of the hollow portion 110.

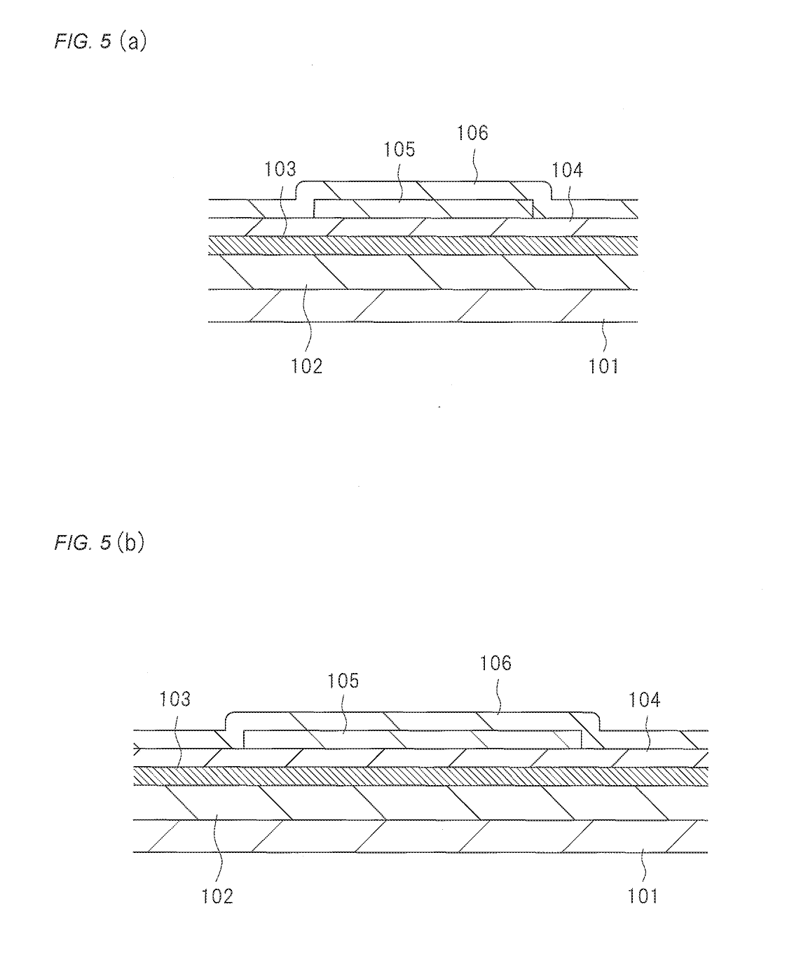

Next, as shown in FIGS. 5(a) and 5(b), the insulating film 106 made of a silicon oxide film having a film thickness of about 200 nm is deposited on upper parts of the insulating film 104 and the sacrificial layer 105 by the plasma CVD method.

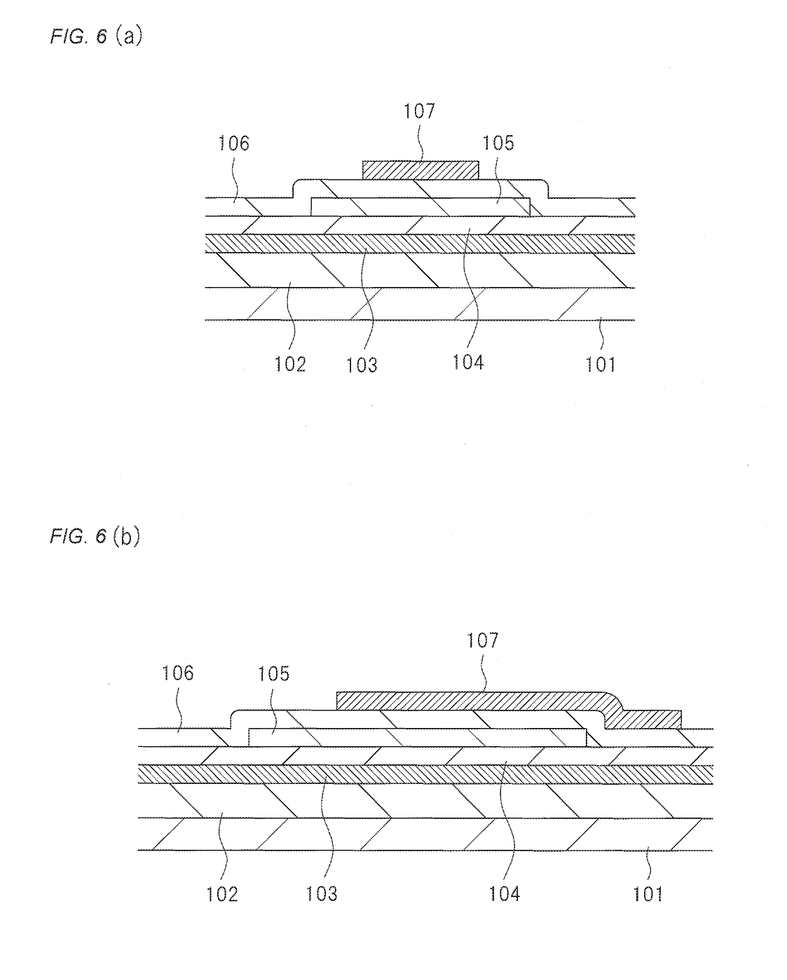

Next, as shown in FIGS. 6(a) and 6(b), an aluminum alloy film having a film thickness of about 100 nm is deposited on an upper part of the insulating film 106 by the sputtering method, and then the aluminum alloy film is patterned by using the photolithography technique and the dry etching technique to form the upper electrode 107.

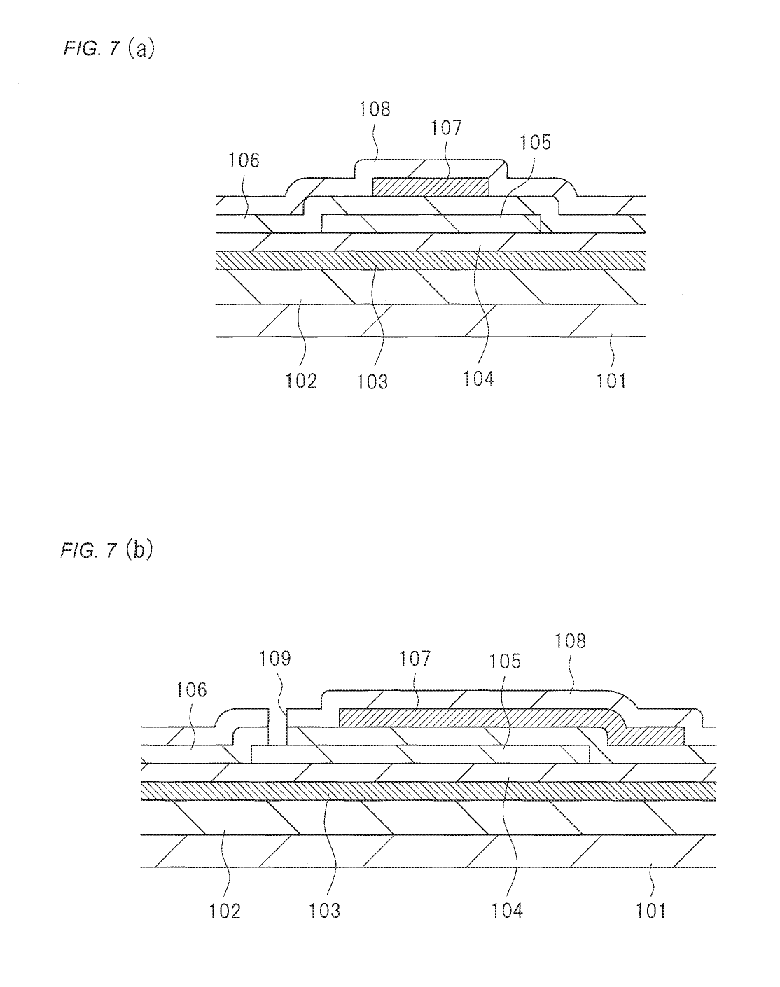

Next, as shown in FIGS. 7(a) and 7(c), the insulating film 108 made of a silicon oxide film having a film thickness of about 200 nm is deposited on upper parts of the insulating film 106 and the upper electrode 107 by the plasma CVD method, and then a part of each of the insulating films 108 and 106 is removed by using the photolithography technique and the dry etching technique, thereby forming an opening 109 which reaches the sacrificial layer 105.

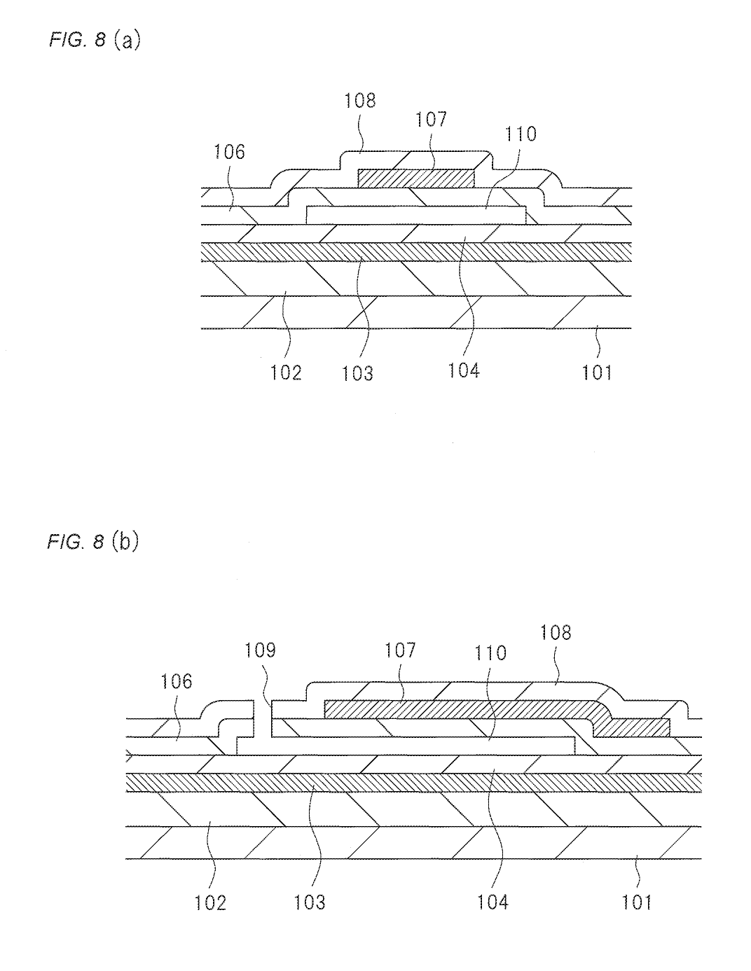

Next, as shown in FIGS. 8(a) and 8(b), the sacrificial layer 105 is dissolved by using wet etching solution such as a potassium hydroxide aqueous solution coming into contact with a surface of the sacrificial layer 105 through the opening 109. The hollow portion 110 is thereby formed in a region where the sacrificial layer 105 was formed.

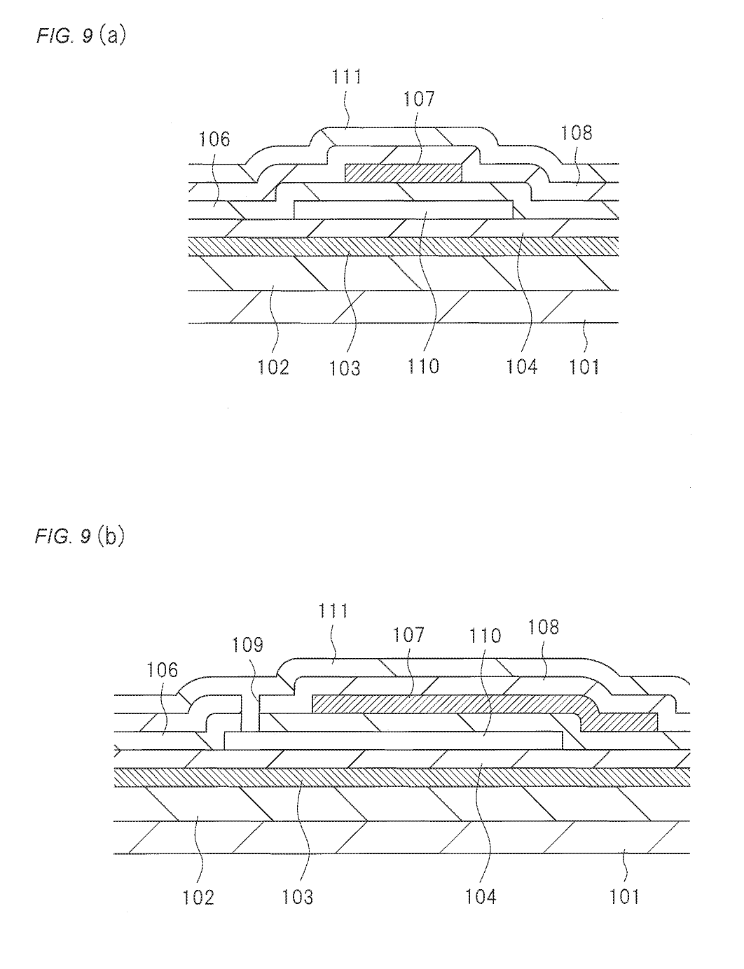

Next, as shown in FIGS. 9(a) and 9(b), the insulating film 111 made of a silicon oxide film having a film thickness of about 500 nm is deposited on an upper part of the insulating film 108 by the plasma CVD method. The insulating film 111 is thereby embedded inside the opening 109, so that the hollow portion 110 is sealed.

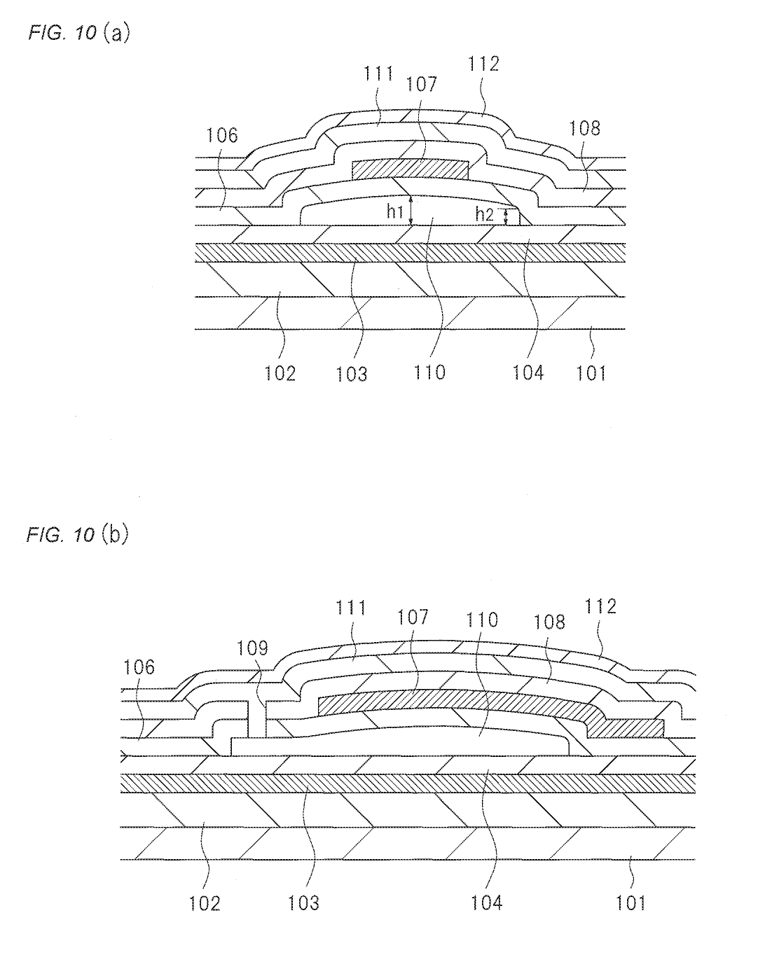

Next, as shown in FIGS. 10(a) and 10(b), the insulating film 112 made of a silicon nitride film having a film thickness of about 500 nm is deposited on an upper part of the insulating film 111 by the plasma CVD method. Since the silicon nitride film constituting the insulating film 112 has a denser film quality than a silicon oxide film, it has a high residual stress. Accordingly, when the insulating film 112 made of a silicon nitride film is deposited on the upper parts of the insulating films 106, 108, and 111 made of silicon oxide films, the residual stress of the insulating film 112 is applied to the insulating films 106, 108, and 111, so that the insulating films 106, 108, and 111 above the hollow portion 110 are pulled upward. As a result, the thickness (h1) of the center portion becomes greater than the thickness (h2) of the side wall portion 118 along the outer peripheral portion, and the hollow portion 110 has the cross-sectional shape according to which the relationship of h1>h2>0 is established.

Then, by using the photolithography technique and the dry etching technique, the connection hole 113 is formed in the insulating films 112, 111, 108, 106, and 104, and the connection hole 114 is formed in the insulating films 112, 111, and 108, so that the pad 115 where a part of the lower electrode 103 is exposed and the pad 116 where a part of the upper electrode 107 is exposed are formed. In this manner, the CMUT shown in FIGS. 1, 2(a), and 2(b) is completed.

Note that the electrode material and the insulating film materials forming the CMUT described above are preferable examples, and are not restrictive. As the electrode material, metal materials other than aluminum alloy, such as W, Ti, TiN, Al, Cr, Pt, Au, may also be used, or polycrystalline silicon doped with impurities at high concentration or amorphous silicon may also be used. Furthermore, a silicon oxynitride film, a hafnium oxide film, a silicon-doped hafnium oxide film or the like may be used instead of the insulating film made of a silicon oxide film. The sacrificial layer 105 is not limited to the polycrystalline silicon film and may be a metal film, a spin-on-glass (SOG) film or the like, as long as it is made of a material having a high etching selectivity with respect to the insulating films.

In the manufacturing method described above, the thickness of the center portion of the hollow portion 110 is made greater than the thickness of the outer peripheral portion (side wall portion 118) by using the residual stress of the silicon nitride film (insulating film 112), but the following method may alternatively be used.

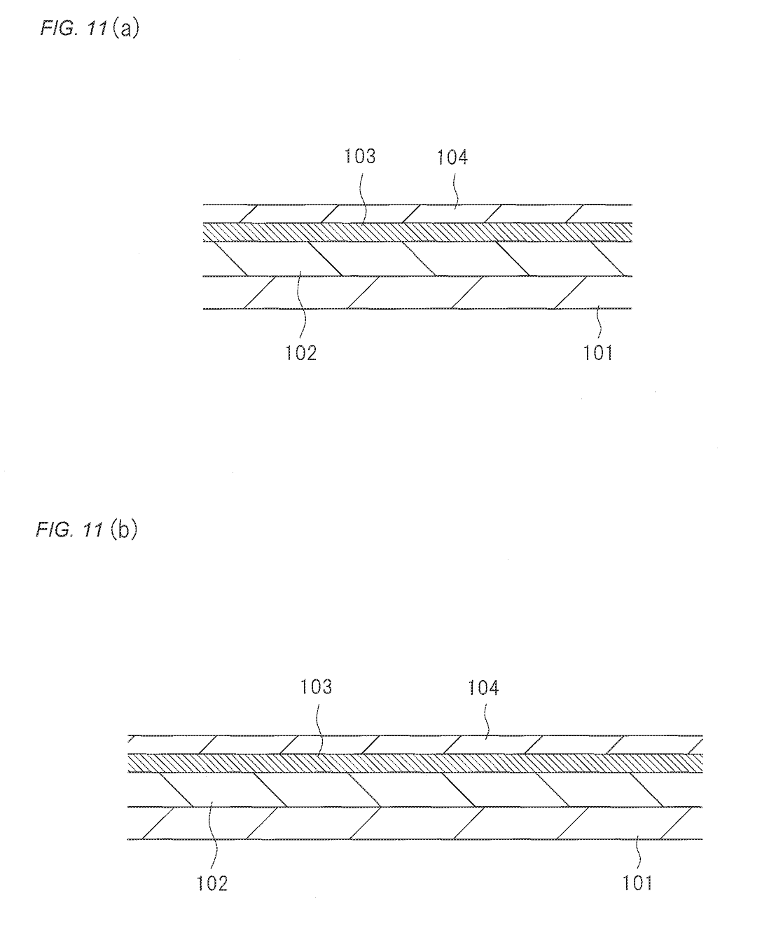

First, as shown in FIGS. 11(a) and 11(b), the insulating film 102, the lower electrode 103, and the insulating film 104 are sequentially formed over the substrate 101 according to the above-described step shown in FIGS. 3(a) and 3(b).

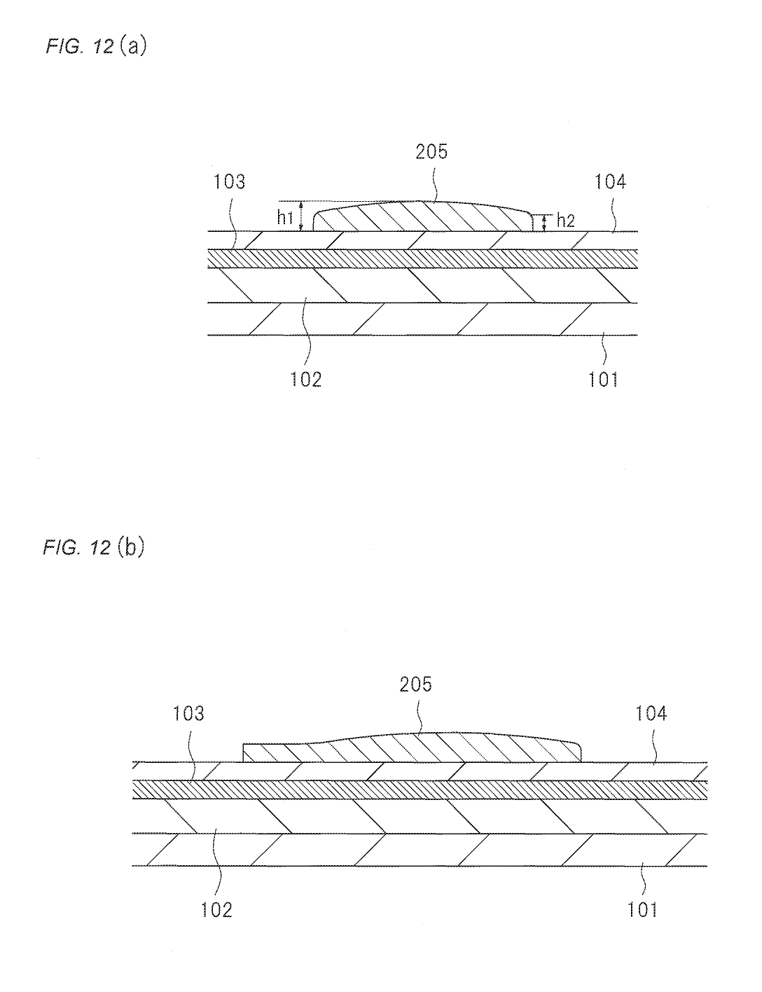

Next, as shown in FIGS. 12(a) and 12(b), after a polycrystalline silicon film having a film thickness of about 200 nm is deposited on the upper part of the insulating film 104 by the plasma CVD method, a sacrificial layer 205 having a cross-sectional shape according to which the thickness (h1) of the center portion is greater than the height (h2) of the outer peripheral portion and the relationship of h1>h2>0 is established is formed by the photolithography technique and the dry etching technique using a grayscale photomask.

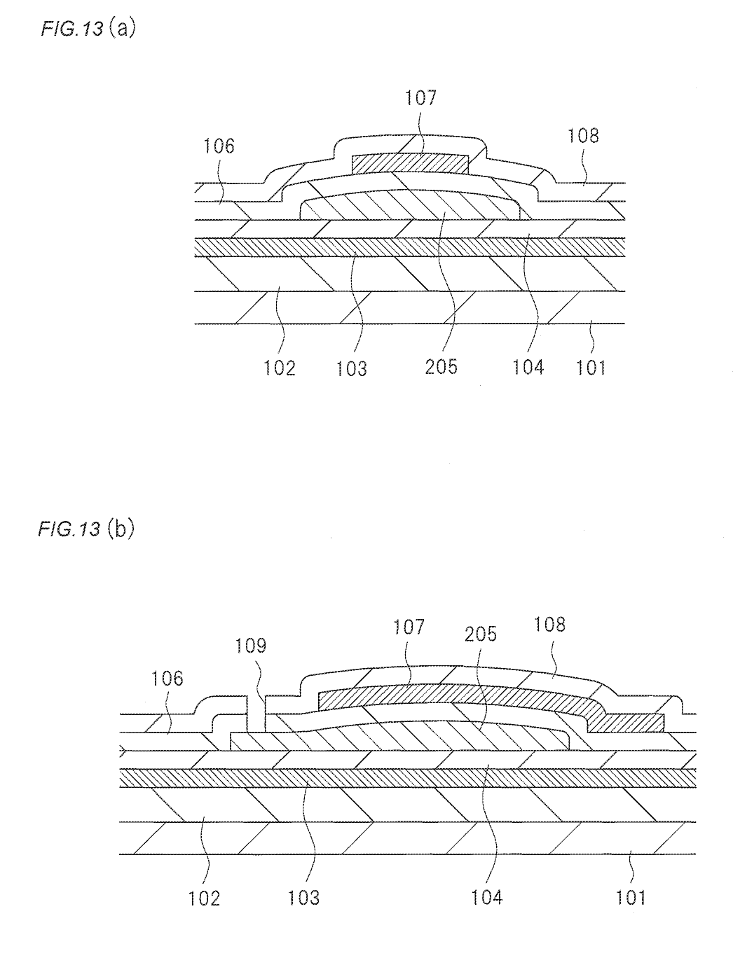

Next, as shown in FIGS. 13(a) and 13(b), after the insulating film 106 is formed on the upper parts of the insulating film 104 and the sacrificial layer 205 according to the above-described steps shown in FIGS. 5 to 7 and the upper electrode 107 and the insulating film 108 are sequentially formed on the upper part of the insulating film 106, a part of each of the insulating films 108 and 106 is removed, thereby forming the opening 109 which reaches the sacrificial layer 205.

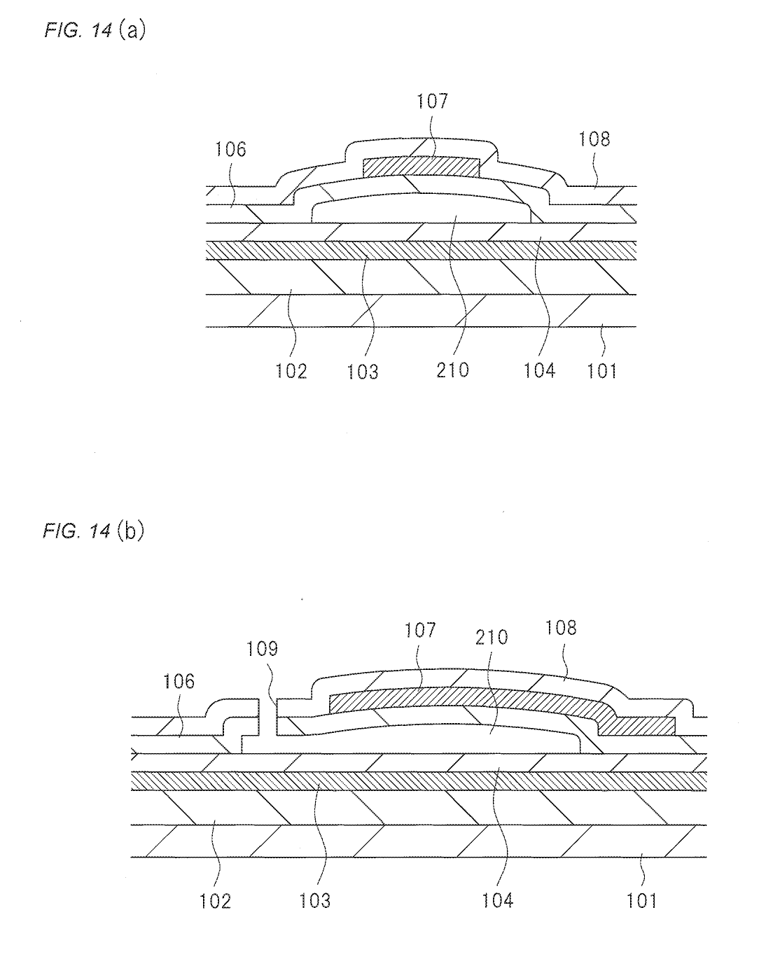

Next, as shown in FIGS. 14(a) and 14(b), according to the above-described step shown in FIG. 8, the sacrificial layer 205 is dissolved by using wet etching solution coming into contact with a surface of the sacrificial layer 205 through the opening 109, thereby forming a hollow portion 210 in a region where the sacrificial layer 205 was formed.

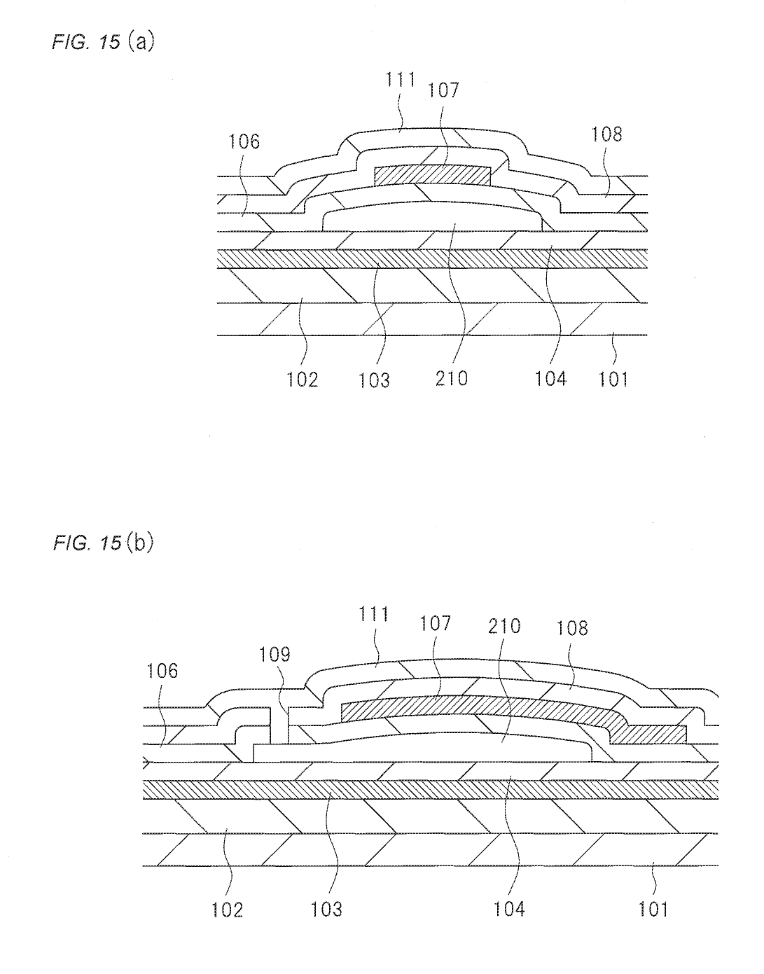

Next, as shown in FIGS. 15(a) and 15(b), the insulating film 111 is deposited on the upper part of the insulating film 108 according to the above-described step shown in FIG. 9, so that the insulating film 111 is embedded inside the opening 109 and the hollow portion 210 is sealed.

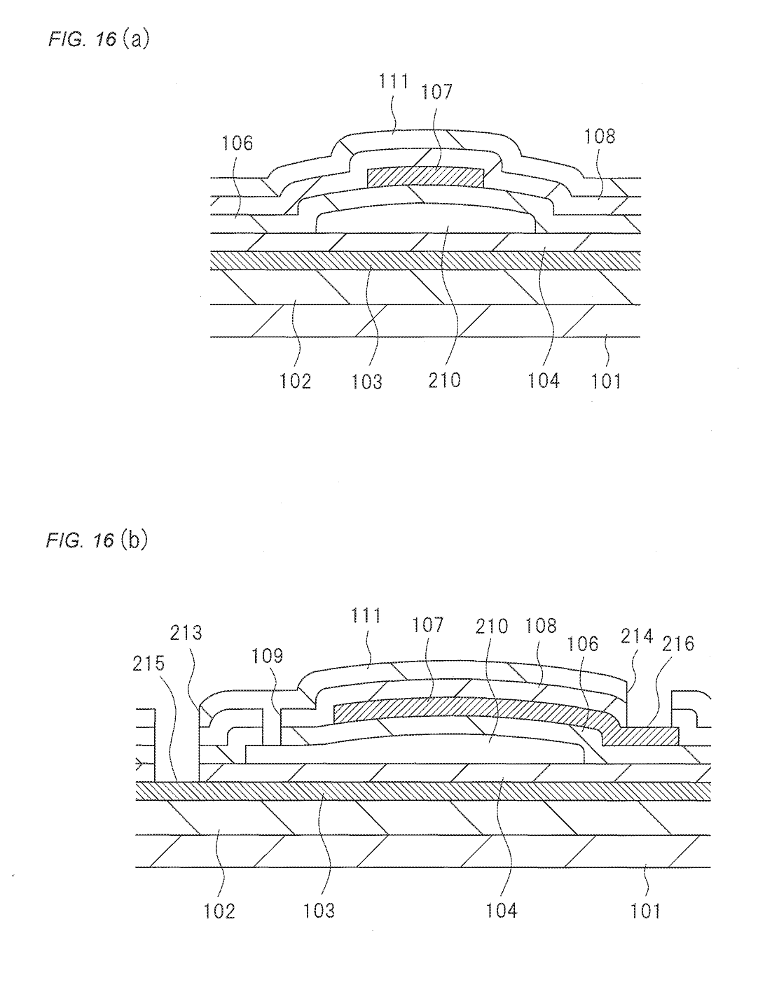

Then, as shown in FIGS. 16(a) and 16(b), pads 215 and 216 are formed by forming a connection hole 213 in the insulating films 111, 108, 106, and 104 and forming a connection hole 214 in the insulating films 111 and 108 by using the photolithography technique and the dry etching technique.

In the manufacturing method shown in FIGS. 11(a) to 16(b), the residual stress of a silicon nitride film (insulating film 112) is not used, and thus, the cross-sectional shape of the hollow portion 210 can be defined without any inconveniences caused by the stress such as delamination of insulating films.

In the manufacturing method shown in FIGS. 11(a) to 16(b), similarly, the cross-sectional shape of the hollow portion 210 can be controlled with high accuracy by depositing the insulating film having a high residual stress on the upper part of the insulating film 111. In this case, by using an insulating film having a relatively small residual stress as the insulating film deposited on the upper part of the insulating film 111 or by reducing the film thickness of the insulating film, occurrence of an inconvenience such as delamination of insulating films caused by the stress can be suppressed compared to the case where the cross-sectional shape of the hollow portion 210 is deformed by only the residual stress.

Next, effects of the CMUT of the present embodiment provided with the hollow portion 110 having the cross-sectional shape described above will be described while comparing with a conventional technology.

First, an operation of a CMUT provided with a hollow portion having a rectangular cross-section, that is, a general shape according to which the height is uniform from the center portion to the outer peripheral portion (hereinafter referred to as “basic structure”) will be described.

In this case, when a DC voltage and an AC voltage are superimposed and applied between an upper electrode and a lower electrode, an electrostatic force acts between the electrodes, and a membrane configured of an insulating film and the upper electrode above the hollow portion is elastically deformed and is vibrated according to a frequency of the AC voltage, thereby transmitting ultrasonic wave. Namely, pressure of the ultrasonic wave transmitted from the CMUT is dependent on a vibration amplitude of the membrane. On the other hand, in the case of reception, since the membrane is vibrated by pressure of ultrasonic wave that reaches a surface of the membrane from outside and the distance between the electrodes is changed, the change in the distance is electrically detected as a change in capacitance, thereby receiving ultrasonic wave.

As can be understood from the operation principles described above, the pressure of ultrasonic wave to be transmitted is dependent on the vibration amplitude of the membrane. In the case of the CMUT provided with the hollow portion having a rectangular cross-section, the membrane is supported by a fixing portion (insulating film) at the outer peripheral portion of the hollow portion, and the vibration amplitude occurs by deflection caused by the elastic deformation of the membrane near the center portion of the hollow portion. Accordingly, the vibration amplitude of the membrane has a continuous distribution in which the amplitude is zero at the outer peripheral portion of the hollow portion and is the maximum at the center portion of the hollow portion.

In the CMUT having such an amplitude distribution, a region of the upper electrode near the outer peripheral portion of the hollow portion does not contribute much to the generation of electrostatic force. This is because since the distance between the upper and lower electrodes near the outer peripheral portion of the hollow portion cannot be reduced during vibration, the electrostatic force which is inversely proportional to the square of the distance between the electrodes (in the case where a dielectric is inserted, the distance equivalent to that obtained by conversion to vacuum based on a relative permittivity of the dielectric) is a fraction of the electrostatic force at the maximum amplitude point of the membrane (in other words, a point at which the distance between the electrodes is the smallest).

This property is a major obstacle in increasing the pressure of ultrasonic wave to be transmitted. This is because the maximum amplitude of the membrane needs to be increased or the hollow portion needs to be formed to have a larger height to increase the pressure of ultrasonic wave, but in this case, the membrane has to be vibrated while compensating for the reduction in the electrostatic force caused by the increase in the distance between the electrodes.

Further, it is also possible to increase the electrostatic force by increasing an area of the electrode in an area of the hollow portion, but an effect thereof is limited because the region of the upper electrode near the fixing portion does not contribute much to the generation of the electrostatic force due to the reason given above. In addition, since it leads to the increase in electrostatic capacitance components which do not contribute to the vibration, that is, the increase in parasitic capacitance, sensitivity at the time of transmission/reception is reduced.

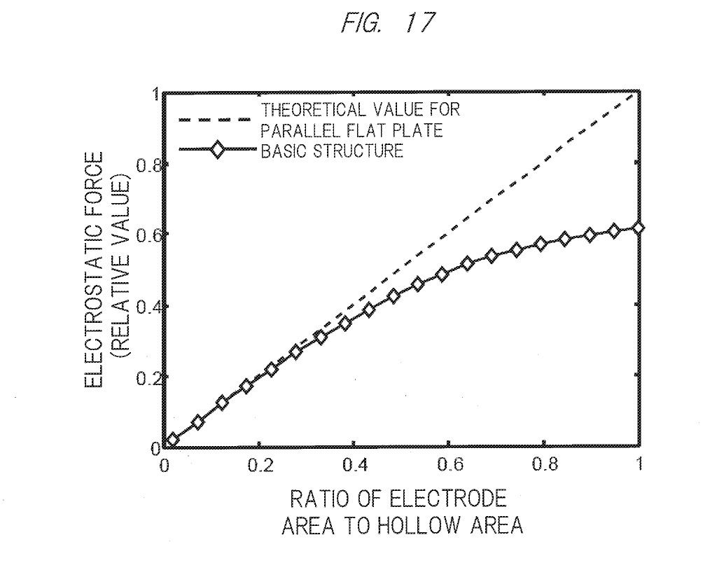

A specific example of an influence on the electrostatic force due to an increase in an electrode area is shown in the graph in FIG. 17. A horizontal axis of the graph represents a ratio of the area of the upper electrode to the area of the hollow portion (hereinafter referred to as “electrode area ratio”), and a vertical axis represents a magnitude of the electrostatic force which is generated when a specific voltage is applied between the electrodes. A plotted broken line in the graph indicates an ideal case, that is, a theoretical value of a change in the electrostatic force generated by a parallel flat plate which moves vertically in a piston-like manner. Also, plotted rhombi indicate a change in the electrostatic force generated by the CMUT provided with the hollow portion having the basic structure described above, that is, a flat membrane.

As indicated by the plotted broken line, in the ideal case, the electrostatic force is simply proportional to the electrode area ratio, and becomes maximum when the electrode area ratio is 100%. On the other hand, as indicated by the plotted rhombi, in the CMUT in which the vibration amplitude has a distribution, the increase in the electrostatic force is slowed when the electrode area ratio exceeds 75%, and the electrostatic force is just 60% of the ideal value at the maximum.

As described above, as long as the CMUT having the basic structure with a flat membrane is used, there is a limit on the increase in the electrostatic force by means of the increase in the electrode area ratio. In order to obtain an electrostatic force sufficient to generate practical sound pressure under such a restriction, a drive voltage has to be increased. However, the high drive voltage is not desirable because it increases an electric field intensity applied between the electrodes, resulting in the occurrence of significant problem of the reduction in reliability such as the breakage of a CMUT element and the faster progress in property deterioration.

The breakage and property deterioration in the CMUT are mainly caused by deterioration of insulating films above and below the hollow portion. These insulating films are formed to separate the lower electrode and the upper electrode and to prevent breakage due to a short-circuit current, but when excessively strong electric field is applied to these insulating films, this may lead to problems such as occurrence of dielectric breakdown and occurrence of charge-up of the insulating films due to injection of charges from the electrodes to the insulating films. When the dielectric breakdown is caused, Joule heat is generated due to increased current, and the CMUT element is broken and becomes unusable. Also, when the charge-up of the insulating film occurs, an electric field between the upper and lower electrodes is shielded by the charges in the insulating film, and a problem that optimal driving cannot be performed arises.

As described above, in the CMUT having the basic structure provided with the hollow portion having the rectangular cross-section, it is difficult to reduce a drive voltage that is necessary to vibrate the membrane.

On the other hand, in the CMUT of Patent Document 1 in which the height of the hollow portion is made zero at the outer peripheral portion of the hollow portion, since the distance between the electrodes is reduced near the outer peripheral portion of the hollow portion, the region of the hollow portion near the outer peripheral portion can also contribute to the generation of an electrostatic force, so that the membrane can be driven at a lower drive voltage.

However, the structure disclosed in Patent Document 1 in which a distribution of the height of the hollow portion is the maximum at the center portion of the hollow portion and is zero at the outer peripheral portion of the hollow portion (Bessel function of the zeroth order, arc function, and sine function are disclosed as examples) has a problem that reliability is reduced in the ultrasonic transmission at a large amplitude.

Namely, in the hollow portion whose height at the outer peripheral portion is zero as in the CMUT disclosed in Patent Document 1, the insulating films above and below the hollow portion come into contact with each other not only at the center portion of the hollow portion but also at the outer peripheral portion when the membrane is vibrated to the most, and thus, a contact area between the insulating layers above and below the hollow portion is increased compared with a hollow portion having the general cross-sectional shape described above. As a result, since charge-up of the insulating films above and below the hollow portion is likely to occur in the case of use over a long period of time, charges between the upper and lower electrodes are shielded by the charges trapped in the insulating layers, so that appropriate driving cannot be performed or the dielectric breakdown may be easily caused in the insulating layers.

In contrast, the hollow portion 110 of the CMUT of the present embodiment has a cross-sectional shape according to which the relationship of h1>h2>0 is established when the thickness of the center portion is given as h1 and the thickness of the side wall portion 118 provided at the outer peripheral portion is given as h2 when a voltage is not applied between the lower electrode 103 and the upper electrode 107.

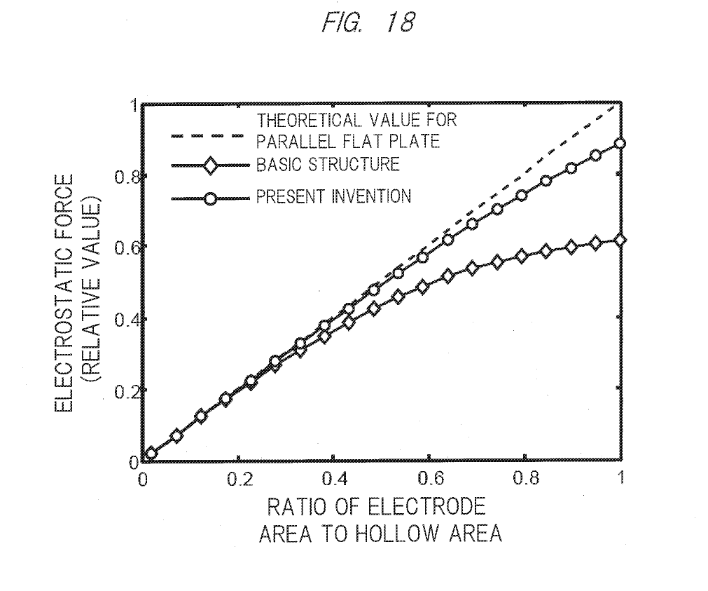

An effect on an increase in the electrostatic force in the case where the hollow portion 110 is configured to have the cross-sectional shape described above is shown in the graph in FIG. 18. Examples of numerical values shown in FIG. 17 are also shown in the graph for comparison. Namely, the plotted broken line indicates an ideal case, that is, a theoretical value of a change in the electrostatic force generated by a parallel flat plate which moves vertically in a piston-like manner, and the plotted rhombi indicate a change in the electrostatic force generated by the CMUT provided with the hollow portion having the rectangular cross-section described above. Further, plotted circles indicate a change in the electrostatic force generated by the CMUT of the present embodiment.

As described above, in the case of the CMUT provided with the hollow portion having a rectangular cross-section, an increase in the electrostatic force is slowed when the electrode area ratio exceeds 75%, and the electrostatic force is just 60% of the ideal value at the maximum. On the other hand, in the case of the CMUT of the present embodiment, the electrostatic force is significantly increased even at the electrode area ratio of 75% or more in which the increase in electrostatic force is not expected in the CMUT provided with the hollow portion having a rectangular cross-section, and the electrostatic force equal to 90% of the ideal value can be achieved. The fact that the strong electrostatic force can be generated at the same voltage means that the same electrostatic force can be generated at a lower voltage.

Further, since the hollow portion 110 has a certain thickness (h2) near the side wall portion 118 in the CMUT of the present embodiment, in a state where displacement is so small that the insulating films between the upper and lower electrodes do not come into contact with each other, a large electrostatic force is generated in regions of the upper and lower electrodes near the side wall portion 118, and in a state where displacement is so large that the insulating films between the electrodes may come into contact with each other, only regions of the insulating films near the center of the hollow portion 110 where the vibration amplitude is the maximum come into contact with each other. Since the hollow portion near the side wall portion 118 has a certain height, contact parts of the insulating films can be limited, and by arranging a structure for reducing the electric field intensity at the contact parts, a measure against electric field concentration caused by contact between the insulating films can be implemented only at the center portion of the hollow portion, so that deterioration of the insulating films can be suppressed.

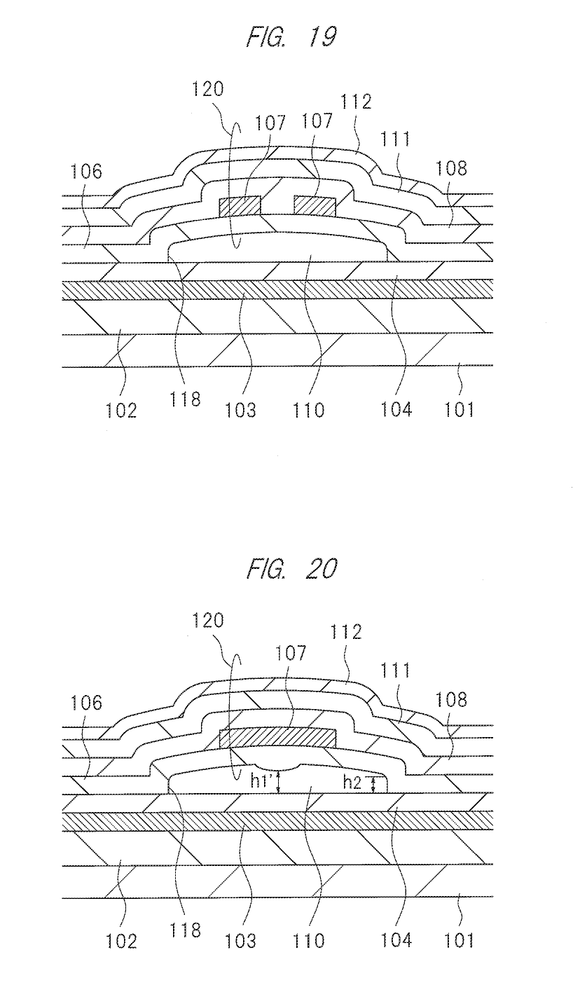

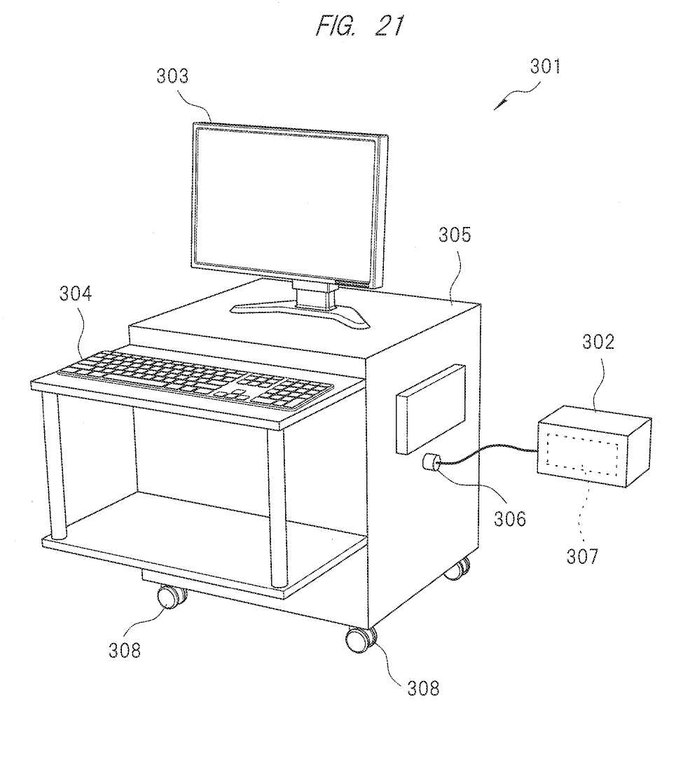

As specific measures taken against electric field concentration, for example, the following methods are conceivable. FIG. 19 is an example where concentration of the electric field is suppressed by removing an electrode part of at least one of the lower electrode 103 and the upper electrode 107 at a region where the insulating films 104 and 106 come into contact with each other. Also, FIG. 20 is an example where the electric field intensity is reduced to a level where accumulation of charges does not become a problem even if the insulating films 104 and 106 come into contact with each other, by locally increasing the film thickness of at least one of the insulating films 104 and 106 at a region where the insulating films 104 and 106 come into contact with each other. Even in the case of adopting the method in FIG. 20, it is necessary to form the cross-sectional shape according to which a relationship of h1′>h2>0 is established when the thickness of the center portion is given as h1′ and the thickness of the side wall portion 118 provided at the outer peripheral portion is given as h2 when a voltage is not applied between the lower electrode 103 and the upper electrode 107.

In the example in FIG. 19, the electrode part is removed at a maximum displacement part of the membrane 120, but the electrode part may be removed at several positions of the membrane 120. Further, in the example in FIG. 20, the thickness of the insulating film is increased at the maximum displacement part of the membrane 120, but the insulating film may be made thick at several positions of the membrane 120.

As described above, according to the present embodiment, a CMUT capable of achieving both a reduced drive voltage and long-term reliability can be realized.

### Second Embodiment

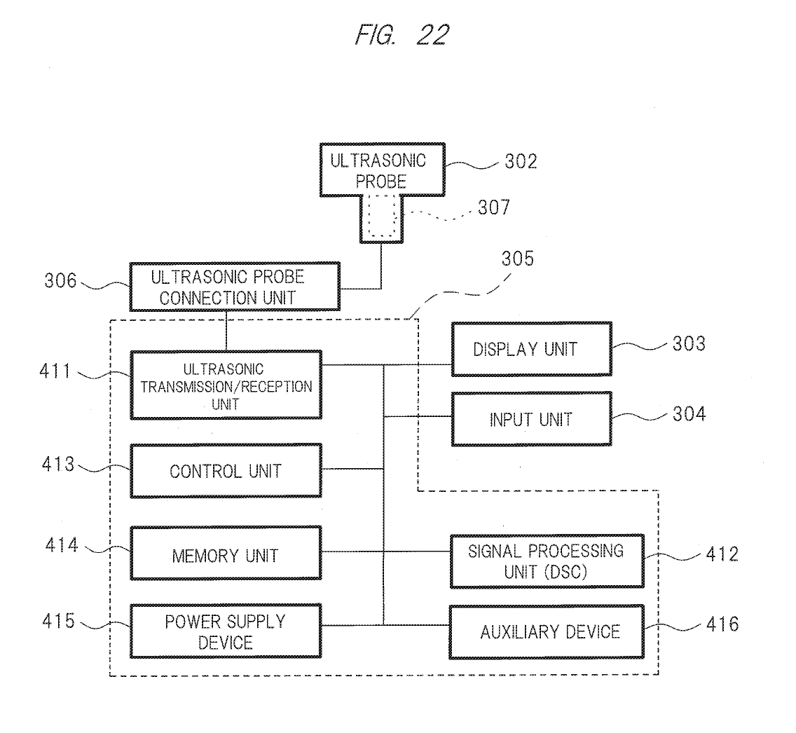

FIG. 21 is a perspective view showing an external appearance of an ultrasonic imaging device provided with the CMUT of the first embodiment, and FIG. 22 is a block diagram showing functions of the ultrasonic imaging device shown in FIG. 21.

An ultrasonic imaging device 301 includes: a main body 305 which houses an ultrasonic transmission/reception circuit for transmitting and receiving ultrasonic wave, a signal processing circuit for processing an echo signal received by the ultrasonic transmission/reception circuit and generating an ultrasonic image of a target to be inspected, and the like; a display unit 303 which is connected to the main body 305 and displays an ultrasonic image and a GUI as an interface to an operator; an input unit 304 which is operated by the operator; and an ultrasonic probe 302 which is connected to the ultrasonic transmission/reception circuit through an ultrasonic probe connection unit 306 fixed to the main body 305.

The ultrasonic probe 302 is a device for transmitting and receiving ultrasonic wave to and from a subject (patient) by coming into contact with the subject, and includes an ultrasonic transducer 307 having a structure where a large number of transducer elements are arranged in a one-dimensional or two-dimensional array, an acoustic lens, a packing material, and the like. The ultrasonic transducer 307 is configured by arranging about several hundred to ten thousand CMUT elements in a one-dimensional or two-dimensional array.

Although FIG. 21 shows a movable ultrasonic imaging device having casters 308 provided on a bottom portion of the main body 305 as an example, the ultrasonic imaging device 301 of the present embodiment can be applied to an ultrasonic imaging device which is fixed in an inspection room, a portable ultrasonic imaging device of a notebook type or a box type, or the other known ultrasonic imaging devices.

As shown in FIG. 22, the main body 305 of the ultrasonic imaging device 301 includes an ultrasonic transmission/reception unit 411, a signal processing unit 412, a control unit 413, a memory unit 414, a power supply device 415, and an auxiliary device 416.

The ultrasonic transmission/reception unit 411 is configured to generate a drive voltage for transmitting ultrasonic wave from the ultrasonic probe 302 and receive an echo signal from the ultrasonic probe 302, and includes a delay circuit, a filter, a gain adjustment circuit, and the like.

The signal processing unit 412 is configured to perform processing necessary for correction and image creation such as LOG compression and depth compensation on a received echo signal, and may include a digital scan converter (DSC), a color Doppler circuit, an FFT analysis unit, and the like. As signal processing by the signal processing unit 412, both analog signal processing and digital signal processing are possible, and the signal processing can be partially realized by software or can be realized by an application specific integrated circuit (ASIC) or a field-programmable gate array (FPGA).

The control unit 413 controls each circuit in the main body 305 and appliances connected to the main body 305. Information and parameters necessary for signal processing and control and processing results are stored in the memory unit 414. The power supply device 415 supplies necessary power to each unit of the ultrasonic imaging device. The auxiliary device 416 is provided for realizing functions accompanied with each unit described above in the ultrasonic imaging device 301, such as audio generation, and is added as appropriate when needed.

Since the ultrasonic imaging device 301 of the present embodiment uses the CMUT of the first embodiment described above as the ultrasonic transducer 307 of the ultrasonic probe 302, it is possible to transmit and receive ultrasonic wave with high sensitivity at a low voltage which is safe even when a subject (patient) comes into contact. Furthermore, since the long-term reliability of the CMUT is high, it is possible to reduce running costs of the ultrasonic imaging device 301.

In the foregoing, the invention made by the present inventors has been specifically described based on the embodiments, but the present invention is not limited to the embodiments described above, and various modifications are possible within the scope not departing from the gist of the invention.

### REFERENCE SIGN LIST

101 substrate
102 insulating film
103 lower electrode
104 insulating film
105 sacrificial layer
106 insulating film
107 upper electrode
108 insulating film
109 opening
110 hollow portion
111 insulating film
112 insulating film
113 connection hole
114 connection hole
115 pad
116 pad
118 side wall portion
120 membrane
205 sacrificial layer
210 hollow portion
213 connection hole
214 connection hole
215 pad
216 pad
301 ultrasonic imaging device
302 ultrasonic probe
303 display unit
304 input unit
305 main body
306 ultrasonic probe connection unit
307 ultrasonic transducer
308 caster
411 ultrasonic transmission/reception unit
412 signal processing unit
413 control unit
414 memory unit
415 power supply device
416 auxiliary device

1. An ultrasonic transducer comprising:
a substrate;
a lower electrode formed on the substrate;
a hollow portion provided between first and second insulating films that are sequentially formed over the lower electrode;
an upper electrode formed on the second insulating film above the hollow portion;
third and fourth insulating films that are sequentially formed over the upper electrode; and
a membrane formed of the second insulating film, the upper electrode, and the third and fourth insulating films above the hollow portion,
wherein the hollow portion has a cross-sectional shape according to which a relationship of h1>h2>0 is established when a thickness of a center portion is given as h1 and a thickness of an outer peripheral portion is given as h2.

2. The ultrasonic transducer according to claim 1,
wherein a thickness of the hollow portion is monotonically reduced in a curved manner from the center portion toward the outer peripheral portion.

3. The ultrasonic transducer according to claim 1,
wherein a thickness of the hollow portion is linearly reduced from the center portion toward the outer peripheral portion.

4. The ultrasonic transducer according to claim 1,
wherein the hollow portion has a recessed part or a protruding part on a side of the second insulating film above the hollow portion, and
a thickness of the hollow portion is reduced in a curved manner from the center portion toward the outer peripheral portion.

5. The ultrasonic transducer according to claim 1,
wherein a bottom surface of the hollow portion is flat.

6. The ultrasonic transducer according to claim 1,
wherein the thickness (h1) of the center portion is at least 1.5 times the thickness (h2) of the outer peripheral portion.

7. The ultrasonic transducer according to claim 1,
wherein an area of a region where the hollow portion and the upper electrode overlap each other in plan view is at least 75% of an area of the hollow portion.

8. The ultrasonic transducer according to claim 1, further comprising:
a fifth insulating film formed over the fourth insulating film and having a higher residual stress than the second, third and fourth insulating films.

9. A manufacturing method of an ultrasonic transducer, comprising the steps of:
(a) sequentially forming a lower electrode and a first insulating film on a substrate;
(b) forming a sacrificial layer on the first insulating film;
(c) forming a second insulating film that covers the first insulating film and the sacrificial layer;
(d) forming an upper electrode on the second insulating film;
(e) forming a third insulating film that covers the second insulating film and the upper electrode;
(f) removing apart of each of the third and second insulating films to form an opening that reaches a surface of the sacrificial layer;
(g) forming a hollow portion between the first and second insulating films by removing the sacrificial layer through the opening;
(h) forming a fourth insulating film over the third insulating film, thereby embedding the fourth insulating film in the opening; and
(i) depositing, over the fourth insulating film, a fifth insulating film having a higher residual stress than the second, third and fourth insulating films to pull upward the second insulating film, the upper electrode, and the third and fourth insulating films above the hollow portion, thereby making a thickness of a center portion of the hollow portion greater than a thickness of a side wall portion along an outer peripheral portion of the hollow portion.

10. The manufacturing method of an ultrasonic transducer according to claim 9,
wherein the sacrificial layer formed on the first insulating film in the step (b) has a cross-sectional shape according to which a relationship of h1>h2>0 is established when a thickness of a center portion of the sacrificial layer is given as h1 and a thickness of an outer peripheral portion of the sacrificial layer is given as h2.

11. The manufacturing method of an ultrasonic transducer according to claim 10,
wherein in the step (g), an etching solution is supplied to the sacrificial layer through the opening, thereby dissolving and removing the sacrificial layer.

12. An ultrasonic imaging device comprising an ultrasonic probe including the ultrasonic transducer according to claim 1.
